(19) United States
(12) Patent Application Publication
(10) Pub. No.: US 2006/0065267 A1
(43) Pub. Date: Mar. 30, 2006

(54) Breath enhanced nebulizer with interchangeable top portions

(75) Inventors: Tuan Quoc Tran, Glen Allen, VA (US); Lawrence A. Weinstein, Chesterfield, VA (US)

Correspondence Address: MCGUIREWOODS, LLP, 1750 TYSONS BLVD, SUITE 1800, MCLEAN, VA, 22102, US

(73) Assignee: Pari Innovative Manufacturers, Inc., Midlothian, VA (US)

(21) Appl. No.: 11/265,175

(22) Filed: Nov. 3, 2005

Related U.S. Application Data
(63) Continuation-in-part of application No. 10/802,745, filed on Mar. 18, 2004.
(60) Provisional application No. 60/682,380, filed on May 19, 2005.

(51) Int. Cl. A61M 11/00

(52) U.S. Cl. 128/200.14

(57) ABSTRACT

A mouthpiece which is attachable to nebulizer is configured to enhance the flow of gas carrier through an aerosolization portion of the nebulizer. The mouthpiece has a hollow body attached to a drug delivery conduit. The drug delivery conduit has a drug delivery port in one end. The interior of the hollow body is configured to efficiently direct gas carrier flow into the aerosolization chamber of the nebulizer to allow better mixing of the aerosolized substance and the gas carrier before the gas carrier exits the nebulizer. A cap may be removable and replaceable from a hollow body to allow use of the nebulizer in both a breath enhanced setting and a ventilator setting. Further, a cap may be configured to reduce or eliminated spillage from the mouth piece.

### CROSS-REFERENCE TO RELATED APPLICATIONS

This application is a continuation-in-part application of U.S. application Ser. No. 10/802,745, filed Mar. 18, 2004, the disclosure of which is expressly incorporated by reference herein in its entirety. This application further claims priority under 35 U.S.C. §119(c) of the U.S. Provisional Application Ser. No. 60/682,380, filed on May 19, 2005, the disclosure.

### FIELD OF THE INVENTION

The invention relates to a drug delivery device, and more particularly to an interchangeable nebulizer mouthpiece for delivering drugs to a patient.

### BACKGROUND

Drugs and other substances can be administered to a patient through a variety of methods. For example, one method includes intravenous injection where the drug or substance is injected into a person's veins. Another method includes a sublingual delivery system where the drug or substance is placed under the user's tongue and is allowed to be absorbed through the mucus membranes of the mouth. Still another method of drug or substance delivery includes applying the drug or substance directly onto the recipient's skin where it either treats a condition on the surface of the skin or is absorbed into the body.

One method of administering a drug or substance to a patient suffering from respiratory as well as other conditions includes inhaling gas carrier or a gas in which a drug or other substance is dissolved, mixed, entrained, suspended or otherwise incorporated into the gas carrier or gas. With this delivery method, the gas carrier or gas functions as a gas carrier which carries the drug or substance into the user's lungs where is may be absorbed to treat a condition of the lungs or in some instances be transferred into the circulation system of the user to be distributed throughout the body.

It should be noted that although a drug is typically the material which is delivered in this way, virtually any chemical or substance either in a gaseous, liquid, or solid form may be delivered using this inhalation technique. Furthermore, the gas carrier which is typically gas carrier may also be made up only of, or partially of, any kind of gas or mixture of gases where the gas or mixture of gases serves to not only transport the drug or substance into the users lungs, but may also interact with the users tissues as well as interact with the drug or substance being delivered. Such interaction may further enhance the chemical or medical activity of the drug or substance, as well as suppress or enhance certain responses of the tissue to the drug or substance to be delivered.

The drug or substance to be delivered can be incorporated into the gas carrier, which will be referred to as gas carrier hereinafter for simplicity, through a variety of methods. For example, the drug can take the form of a gas and be mixed into the gas carrier and remain suspended in the gas carrier, or dissolved in the gas carrier. Additionally, the delivered substance may exist initially in the form of a bulk liquid and be converted to small droplets or a mist which is mixed or entrained into the gas carrier for transport into the lungs. Furthermore, the delivered substance may be converted from a liquid into a vapor through heating, for example, for incorporation into the gas carrier. Also, the delivered substance may exist in solid form as particles which are mixed, entrained, or otherwise suspended in the gas carrier while the gas carrier is inhaled by the user.

Because the delivered substance may exist in a variety of forms all of which have different degrees of ease of incorporation into the gas carrier flowing into a user's lungs, an efficient means for incorporating the delivered substance into the gas carrier is needed. A typical way of incorporating the delivered substance into a gas carrier is an aerosolization chamber, or nebulizer.

Additionally, the delivered substance can exist in a first form which is then mixed with a carrier substance such as mixing solid particles into a liquid and the resulting liquid-particle mixture is then converted into a mist and incorporated into the gas carrier.

Typically, a nebulizer uses the flow of the gas carrier through a chamber to cause turbulence between the gas carrier and the substance at their interface in order to form a mixture of the substance and gas carrier, the delivered substance is then carried into the user's lungs along a flow path from the mixing area to the user's mouth or nose.

Accordingly, mouthpiece is typically joined to the nebulizer which is then inserted into the patient's mouth during inhalation through the mouth. As such, as the user inhales, the gas carrier is drawn through the mouthpiece and through the nebulizer which initiates the mixing of the delivered substance in the gas carrier.

Because some delivered substances may be difficult to incorporate into a gas carrier, and other delivered substances must be delivered at dosage rates which require the gas carrier to carry a large amount of the delivered substance, it is important that the nebulizer and mouthpiece work to efficiently mix the delivered substance into the gas carrier. Furthermore, because some patients being treated using this inhalation technique are in a weakened or otherwise impgas carriered condition and cannot inhale the gas carrier with a force sufficient to power the mixing process in the nebulizer, efficient mixing is advantageous.

For the above stated reasons, a mouthpiece is needed which can be attached to a variety of nebulizers which makes efficient use of a gas carrier passing through the mouthpiece and nebulizer to easily incorporate a delivered substance into the gas carrier flow. Furthermore, a mouthpiece which directs gas carrier into and out of a nebulizer in such a way as to efficiently mix a delivered substance into the inhaled gas carrier is needed. Also, a mouthpiece which aids in efficiently mixing a delivered substance into a gas carrier and a nebulizer, and which will attach to a variety of nebulizer styles from various manufacturers is needed.

These and other drawbacks may be present.

### SUMMARY OF THE INVENTION

The invention may address these and other drawbacks.

An aspect of the invention provides a nebulizer with interchangeable caps that allow for use with a patient mouthpiece and with a ventilator line.

Another aspect of the invention provides a nebulizer that reduces spillage when tipped over.

According to an aspect of the invention, a breath enhanced device is provided, including a delivery conduit, a cap, and a plurality of cylinders, where the plurality of cylinders are attached to the cap so that when the cap is attached to a container, a liquid within the container is prevented from flowing through the drug delivery conduit when the cap is in a first position.

The plurality of cylinders may include an inner cylinder, an outer cylinder and an intermediate cylinder located between the outer cylinder and the inner cylinder. The inner cylinder and the outer cylinder may have substantially the same height in relation to the cap, and the intermediate cylinder may have a different height in relation to the cap. The device also may include a hollow chamber having two openings, wherein at least one of the plurality of cylinders may attach the cap to the hollow container at the at least one opening and wherein the liquid may be contained in the hollow chamber. The second opening may be attached to a gas carrier flow so that at least a portion of the liquid is aerosolized into the gas carrier flow and the aerosolized gas carrier flow may be delivered to a subject through the delivery conduit. The liquid may be a drug, or a solution including an active ingredient, such as an anti-inflammatory or an anti-infective.

Another aspect of the invention provides a device for delivery of a compound that includes a hollow body having an airflow inlet for receiving a gas carrier flow and an opening, the hollow body containing a liquid, an aerosol insert interacting with the hollow body to aerosolize the liquid into the gas carrier flow, where the aerosol insert is separate and removable from the hollow body, and a cap including a delivery conduit for directing the aerosolized liquid and the gas carrier, where the cap is separate and removable from the hollow body and the aerosol insert.

The cap may direct the aerosolized liquid and the gas carrier to a mouthpiece, or into a ventilator gas carrier flow. The cap may direct the aerosolized liquid and the gas carrier flow into a subject's lungs. The liquid may be a drug and/or a solution. The solution may include an active ingredient, such as an anti-inflammatory or an anti-infective.

Another aspect of the invention provides a device for delivery of a compound that includes a container holding the compound, the container including an insert and receiving a gas carrier and where the gas carrier interacts with the insert to form a mixture of the gas carrier and the compound. The device also includes a cap having a first cap opening and a second cap opening, the first cap opening engaging said container holding the compound. The device further includes a connector having a first container opening connected to the second cap opening, and a second container opening cooperating to direct the mixture.

The connector may direct the aerosolized liquid and the gas carrier to a mouthpiece. The gas carrier may be received from a ventilator. The connector may direct the mixture into a subject's lungs. The liquid may be a drug and/or a solution. The solution may include an active ingredient, such as an anti-inflammatory or an anti-infective.

Additionally features, advantages, and embodiments of the invention may be set forth or apparent from consideration of the following detailed description, drawings and claims. Moreover, it is to be understood that both the foregoing summary of the invention and the following detailed description are exemplary and intended to provide further explanation without limiting the scope of the invention claimed.

### BRIEF DESCRIPTION OF THE DRAWINGS

The accompanying drawings, which are included to provide a further understanding of the invention, are incorporated in and constitute a part of this specification, illustrate embodiments of the invention and together with the detailed description serve to explain the principles of the invention. No attempt is made to show structural details of the invention in more detail than may be necessary for a fundamental understanding of the invention and the various ways in which it may be practiced. In the drawings:

FIG. 1 is an illustration of an embodiment of the invention;

FIG. 2 is a cutaway view of an embodiment of the invention;

FIG. 3 is a side view of an embodiment of the invention;

FIG. 4 is a ghost view of an embodiment of the invention;

FIG. 5 is a bottom view of an embodiment of the invention;

FIG. 6 is a front view of an embodiment of the invention;

FIG. 7 is a quarter view of an embodiment of the invention;

FIG. 8 is a rear view of an embodiment of the invention;

FIG. 9 is a rear view of an embodiment of the invention;

FIG. 10 is a quarter view of an embodiment of the invention;

FIG. 11 is a bottom view of an embodiment of the invention;

FIG. 12 is a top view of an embodiment of the invention;

FIG. 13 is a ghost view of an embodiment of the invention;

FIG. 14 is a front quarter view of an embodiment of the invention;

FIG. 15 is a rear quarter view of an embodiment of the invention;

FIG. 16 is a bottom view of an embodiment of the invention;

FIG. 17 is a top view of an embodiment of the invention;

FIG. 18 is a rear view of an embodiment of the invention;

FIG. 19 illustrates a side view of a hollow body according to principles of the invention;

FIG. 20 illustrates a bottom view of a hollow body according to principles of the invention;

FIG. 21 illustrates a top view of a hollow body according to principles of the invention;

FIGS. 22 and 23 illustrate a hollow body in use according to principles of the invention;

FIG. 24 illustrates a perspective view of the breath cap;

FIG. 25 illustrates a cut-away view of the breath cap from the bottom;

FIG. 26 illustrates a side view of the breath cap;

FIG. 27 illustrates a bottom view of the breath cap;

FIG. 28 is a perspective view of a ventilator cap;

FIG. 29 is a side view of the ventilator cap;

FIG. 30 is a bottom view of the ventilator cap;

FIG. 31 illustrates a perspective view of an aerosol insert;

FIG. 32 illustrates a side view of the aerosol insert;

FIG. 33 illustrates a front view of the aerosol insert:

FIG. 34 illustrates a bottom view of the aerosol insert; and

FIG. 35 illustrates a top view of the aerosol insert.

### DETAILED DESCRIPTION THE INVENTION

The embodiments of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale; and features of one embodiment may be employed with other embodiments as the skilled artisan would recognize, even if not explicitly stated herein. Descriptions of well-known components and processing techniques may be omitted so as to not unnecessarily obscure the embodiments of the invention.

The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law. Moreover, it is noted that like reference numerals represent similar parts throughout the several views of the drawings.

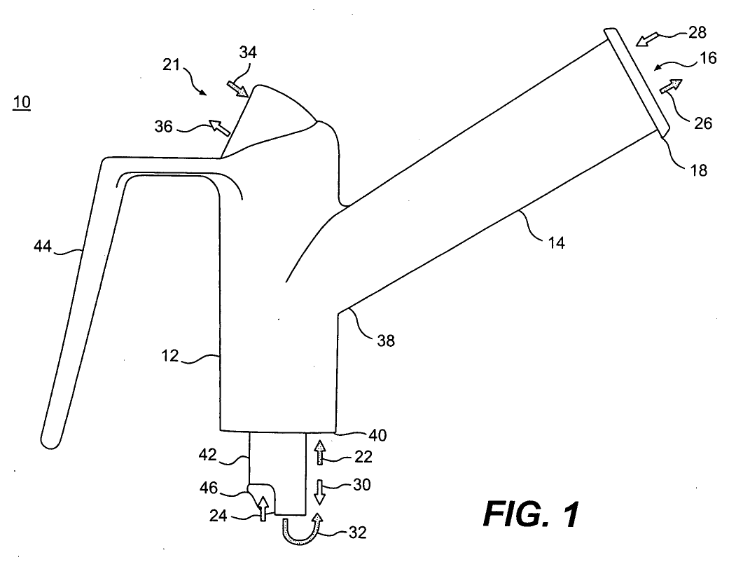

Referring now to the drawings, FIG. 1 is an illustration of an embodiment of a breath enhanced mouthpiece 10. The breath enhanced mouthpiece 10 includes a hollow body 12 also referred to as a body conduit. Attached to the hollow body 12 is a drug delivery conduit 14, also referred to as a drug conveyance. The hollow body 12 and the drug delivery conduit 14 are attached to one another at an intersection 38, also referred to as a union or junction. The drug delivery conduit 14 also includes a user interface 18 which defines a drug delivery port 16 at an end of the drug delivery conduit 14. The drug delivery port 16 can also be referred to as an orifice, opening or aperture, etc.

The drug delivery port 16 defines both an outlet and an inlet into a top flow path. The top flow path at the drug delivery port 16 can be defined as an outflow path 26 upon inhalation and an inflow path 28 upon exhalation, both moving gas carrier through the same volume of drug delivery conduit 14 and through the drug delivery port 16 at different times depending upon whether the patient is inhaling or exhaling.

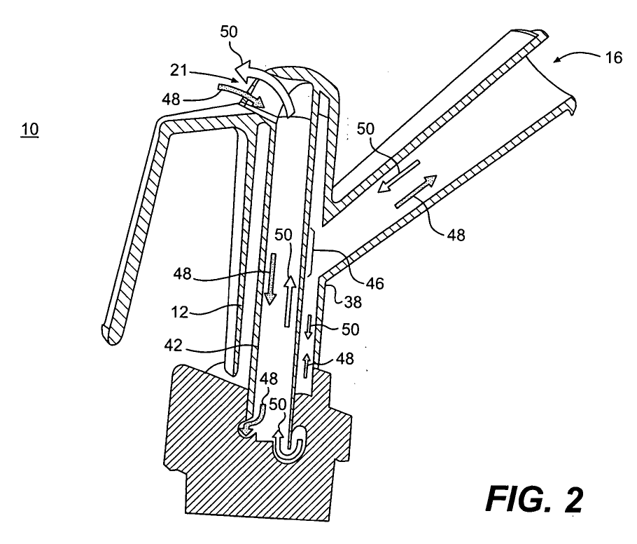

The hollow body 12 has a connection port 40 or opening at its lower end. Within the hollow body 12 is a baffle 42 which can either be planer, circular, tubular, etc. Together, the hollow body 12 and the baffle 42 define two gas carrier flow paths within the hollow body 12. For example, in FIG. 2 the baffle 42 is a tubular conduit. Accordingly, one gas carrier flow path is through the interior of the baffle 42 and another gas carrier flow path is along the exterior of the baffle 42. Both of the aforementioned gas carrier flow paths are through the hollow body 12 of the breath enhanced mouthpiece 10. An end of the baffle 42 may include a notch or cut out 46.

More specifically, the gas carrier flow path through the hollow body 12 may be subdivided into sections. Accordingly, upon inhalation, the gas carrier flow path through the interior of the baffle 42 may include an intake flow path 32, also referred to as a nebulizer inflow path. The gas carrier flow path 32 within the baffle 42 may also include an exhaust gas carrier flow path 24 which occupies the same volume within the baffle 42 upon exhalation. The baffle 42 and the hollow body 12 also define an gas carrier flow path therebetween which include a drug delivery flow path 22, also referred to as an outflow path. Also included in the same volume as this gas carrier flow path is an exhaust flow path 30.

The hollow body 12 has a top which includes a vent 21. The vent 21 may be configured to be selectively sealable. The vent 21 defines one or more gas carrier flow paths through the top of the hollow body 12 including an intake flow path 34 and an exhaust flow path 36. Where the baffle 42 is a conduit, the vent 21 may communicate with an end of that conduit. The hollow body 12 may also include a handle 44 configured to aid in grasping the breath enhanced mouthpiece 10.

In this embodiment of the breath enhanced mouthpiece 10, the drug delivery conduit 14 joins the hollow body 12 at an angle of less than 90 degrees as referenced to the top of the hollow body 12. It should be noted that the drug delivery conduit 14 may join the hollow body 12 at substantially any angle desired, and is preferably configured to be joined at an angle configured for ease of insertion into a user's mouth.

Thus the entire flow path upon inhalation includes flow paths of 22, 26, 32, and 34 of FIG. 1. The entire flow path upon exhalation includes flow paths 28, 30, 24, and 36 of FIG. 1.

As shown in FIG. 1, the mouthpiece 10 is configured to easily attach and detach to other fittings, such as, for example, an aerosolization or nebulization chamber. Accordingly, the mouthpiece 100 may be fitted and removed from a aerosolization chamber at the connection port 40, and thus can be used on a variety of aerosolization chamber, as well as any other type of fitting which may attach at the connection port 40. Thus, although the connection port 40 is shown as a reversible slip or press-fit type of connection, the connection port 40 may be configured to attach by any method well known in the art which allows a detachable or reversible connection to be made, such as threads, a clamp, or a lock.

Referring to FIG. 2, the breath enhanced mouthpiece 10 is shown in a cut away view showing gas carrier flow paths. An entire intake gas carrier flow path 48 shown unsubdivided is represented by the solid arrows and travels from the vent 21 near the top of the hollow body 12 through the interior of the baffle 42 to exit out the bottom of the baffle 42 proximate an aerosolization area. After passing through the aerosolization area, the intake flow path 48 continues between the baffle 42 and the wall of the hollow body 12 to the union 38 between the hollow body 12 and the drug delivery conduit 14.

As shown in FIG. 2, the walls of the hollow body 12 and the walls of the drug delivery conduit 14 define a union inlet 46 at the union 38. The intake flow path 48 passes through the union inlet 46 and proceeds through the drug delivery conduit 14 towards the drug delivery port 16. The intake flow 48 exits the breath enhanced mouthpiece 10 through the drug delivery port 16.

The breath enhanced mouthpiece 10 also defines a second gas carrier flow path also referred to as an exhaust gas carrier flow path 50 represented by the hollow, arrows. It should be noted the exhaust gas carrier flow path 50 may flow along the same volume of flow as the intake flow path 48, however, in the opposite direction thereto and at different times during use of the mouthpiece 10. Accordingly, the exhaust gas carrier flow path 50 flows along a path from the drug delivery port 16 down through the interior of the drug delivery conduit 14 and through the union inlet 46. The exhaust flow path 50 enters the hollow body 12 and flows through the hollow body 12 along the outside of the baffle 42 towards the bottom of the baffle 42. The exhaust flow 50 then enters the bottom of the baffle 42 and flows towards the top of the hollow body 12. Proximate the top of the baffle 42 and hollow body 12, the exhaust flow path 50 exits the hollow body 12 through a vent 21. As can be seen, the intake flow path 48 and the exhaust flow path 50 are substantially along the same path, but opposite of one another.

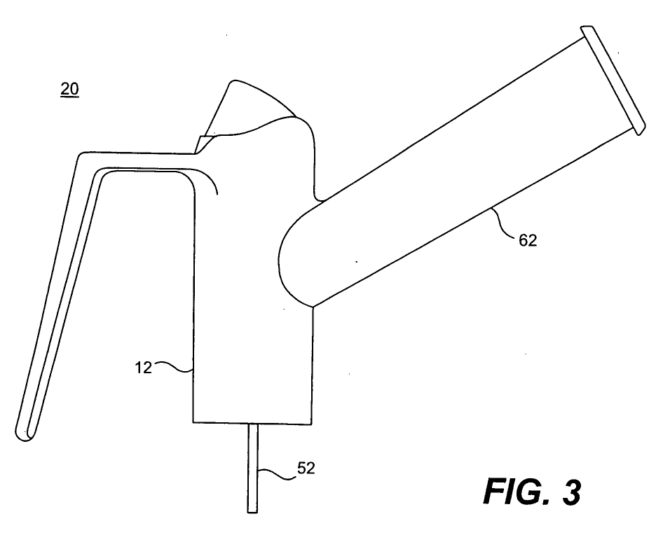

Referring to FIG. 3, another embodiment of the invention is shown as a breath enhanced mouthpiece 20. This embodiment of the breath enhanced mouthpiece 20 has a flat or planer baffle 52 in place of the conduit-like baffle 42 of FIG. 1. As such, the hollow body 12 and the flat baffle 52 define an gas carrier flow path having portions parallel to one another through the interior of the hollow body 12. The baffle 52 may protrude from the bottom of the hollow body 12 with one of its planer surfaces facing towards the drug delivery conduit 14.

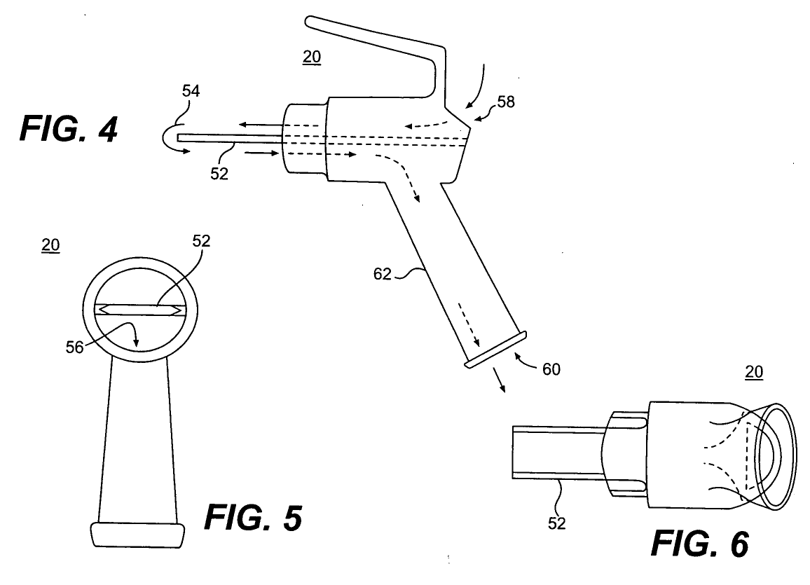

As shown in FIG. 4, the flat baffle 52 and the hollow body 12 define a flow path through the hollow body 12. The flow path extends from a vent 58 at the top of the hollow body 12 and through the interior of the hollow body 12 mounted on one side by the flat baffle 52. The flow path 54 passes around the bottom of the flat baffle 52 and along the opposite side of the flat baffle 52 back into the interior of the hollow body 12. The flow path 54 then passes through the union inlet 56 and into a drug delivery conduit 62 to exit out a drug delivery port 60.

Referring to FIG. 5, a bottom view of the breath enhanced mouthpiece 20 is shown which illustrates how the flat baffle 52 divides the cavity within the hollow body 12 into a first half and a second half where one of the halves contains a union inlet 56 of the breath enhanced mouthpiece 20.

Referring to FIG. 6, the breath enhanced mouthpiece 20 has the flat baffle 52 protruding out of the bottom of the hollow body 12 and has a substantially flat bottom terminating the flat baffle 52. It should be noted that the flat baffle 52 and bottom of the baffle may take on any shape which enhances the efficiency of the mixing process in an attached nebulizer.

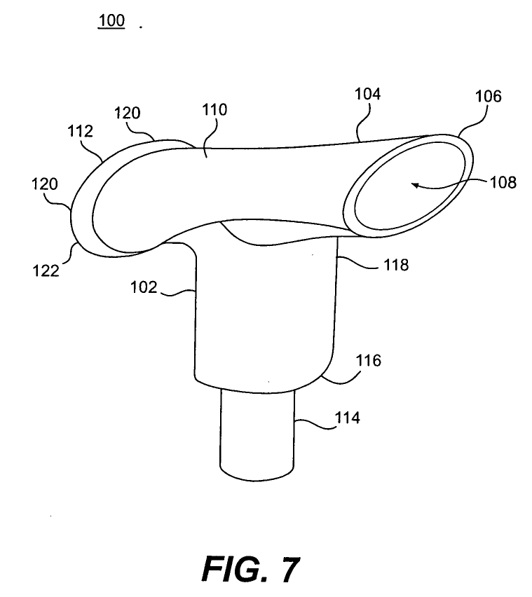

Referring to FIG. 7, a further embodiment of the breath enhanced mouthpiece 100 is shown. This embodiment of the breath enhanced mouthpiece 100 has a hollow body 102 coupled to a drug delivery conduit 104 and a vent conduit 110. The hollow body 102 is joined to the drug delivery conduit 104 and the vent conduit 110 at a junction 118. The junction 118 can also be referred to as a union or an intersection.

The drug delivery conduit 104 includes a user interface 106 and an opening which defines a drug delivery port 108. Vent conduit 110 includes a selectively sealable vent 112 on an end. The hollow body 102 includes a connection port 116 at a lower end of the hollow body 102. Disposed within the hollow body 102 and protruding from the hollow body 102 through the drug connection port 116 is a baffle 114. Although FIG. 7 shows the baffle 114 being circular or tubular, the baffle 114 can be of virtually any shape sufficient to guide a flow of gas carrier including a flat or planar structure. In the embodiment in FIG. 7, the baffle 114 forms a tubular conduit which defines a flow path therein.

The breath enhanced mouthpiece 100 also includes multiple vents on an end of the vent conduit 110. The vent includes a selectively sealable inlet vent 112 and selectively sealable outlet vents 120. Also included on the end of the vent conduit 110 is a diaphragm retainer 122 which is configured to hold a diaphragm on the end of the vent conduit 110.

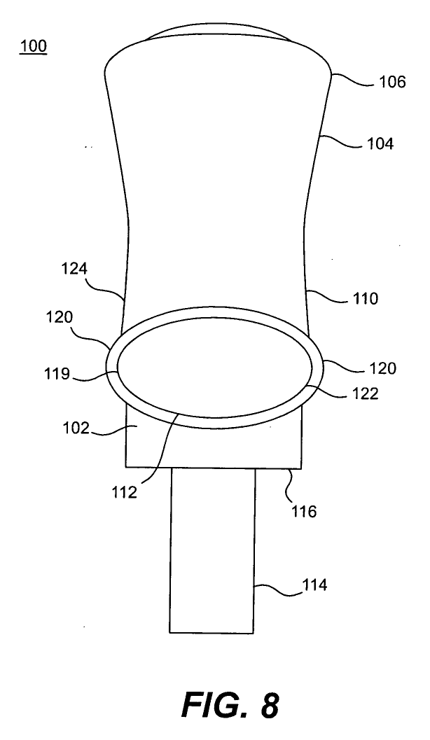

Referring to FIG. 8, a rear view of the breath enhanced mouthpiece 100 is shown. The rear view shows the selectively sealable inlet vent 112 and the selectively sealable outlet vents 119 on either side of the inlet vent 112 on the end of the vent conduit 110. Also shown is the diaphragm retainer 122 and the diaphragm 120. The diaphragm retainer 122 has a diaphragm support 124 which is configured to lie flat across the diaphragm 119. The diaphragm retainer 122 is configured to hold the diaphragm 119 over the end of the vent conduit 110.

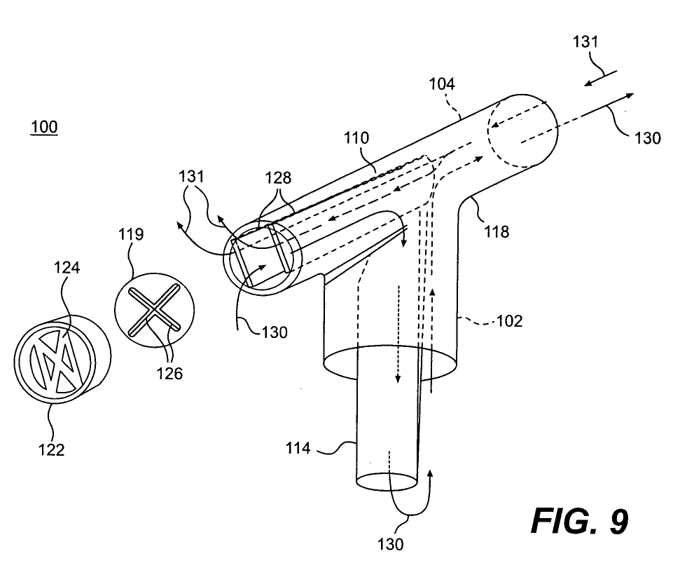

Referring to FIG. 9, an exploded view of the breath enhanced mouthpiece 100 is shown. The vent conduit 110 has walls 128 therein, which divide the interior of the vent conduit 110 into multiple flow paths. The diaphragm 119 is configured to fit over the end of the vent conduit 110 and has slits 126 formed therein. The diaphragm 119 may also have other types of opening beside the slits such as an orifice, aperture, flap, etc. The diaphragm retrainer 122 is configured to fit over the end of the vent conduit 110 and hold the diaphragm 119 in place on the end of the vent conduit 110. As can be seen, the diaphragm retainer 122 has a diaphragm support 124 which are portions of the diaphragm retainer 122 which approximately correspond to the slits 126 in the diaphragm 119.

The structure of the breath enhanced mouthpiece 100 is configured to define various gas carrier flow paths therein. For example, one of the flow paths includes a delivery flow path 130. The delivery flow path 130 may begin at the selectively sealable inlet vent 112 and pass between both walls 128 in the interior of the vent conduit 110. Proximate the union 118, the delivery flow path 130 turns and enters the baffle 114 and passes through the interior of the baffle 114 and out the baffle's lower end. At this point, the delivery flow path 130 may pass through an aerosolization or nebulizing area where drugs or other substances in the forms a gas, a vapor, or small particles or droplets may be entrained, mixed or otherwise incorporated into the gas carrier passing along the flow path 130. The delivery flow path 130 then proceeds along the exterior of the baffle 114 towards the union 118. The delivery flow path 130 enters the drug delivery conduit 104 proximate the union 118, and proceeds along the drug delivery conduit 104 to exit therefrom through the drug delivery port 108.

The breath enhanced mouthpiece 100 includes other flow paths through its interior. For example, an exhaust flow path 131 may include a path from the drug delivery port 108 and through the drug delivery port 104. Proximate the union 118, the exhaust flow path 131 passes into the vent conduit 110 and divides into two paths. Each path passes between a wall 128 and the interior wall of the vent conduit 110. The exhaust flow path 131 then exits the breath enhanced mouthpiece 100 through the selectively sealable output vents 120 past the diaphragm 119.

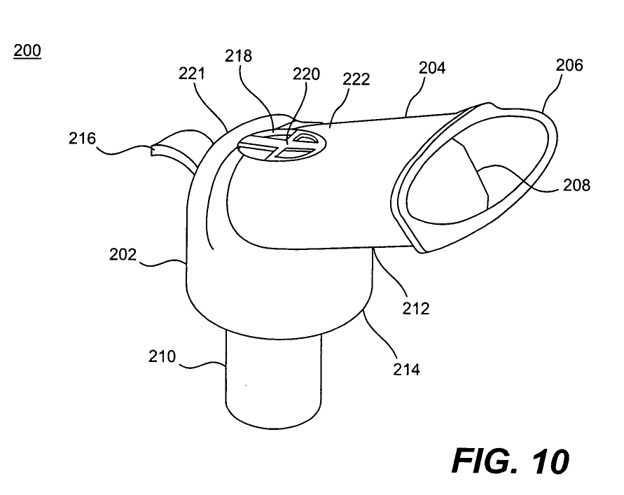

Referring to FIG. 10, a further embodiment of a breath enhance mouthpiece 200 is shown. The breath enhanced mouthpiece 200 includes a hollow body 202 connected to a drug delivery conduit 204 at a junction or union 212. The hollow body 202 includes a connection port 214 proximate its lower portion. Disposed within the hollow body 202 and protruding from the bottom of the hollow body 202 is a baffle 210 which defines a conduit within the baffle.

The drug delivery conduit 204 includes a mouthpiece 206 and a port 208 at an end of the drug delivery conduit 204. Proximate to where the hollow body 202 and drug delivery conduit 204 join at the union 212, is an exhaust aperture 221 covered by a diaphragm 218. The exhaust aperture 221 is located near the top of the hollow body 202. On the exterior of the hollow body 202 near the exhaust aperture 221 is a protrusion 216, which may be used as a hook by which to support the breath enhanced mouthpiece 100 as well as aid a user in holding the breath enhanced mouthpiece 200 during inhalation.

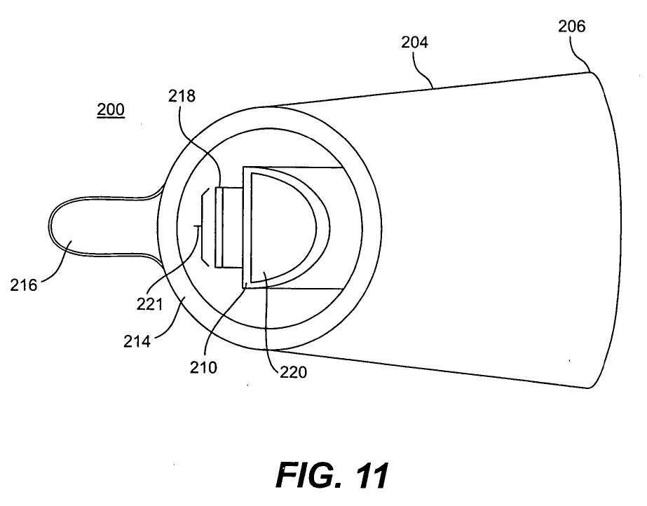

Referring to FIG. 11, a bottom view of the breath enhanced mouthpiece 200 is shown. The bottom of the hollow body 202 terminates in a connection port 214. Disposed within the hollow body 202 is a baffle 210 which, in this example, is a hollow conduit with a perimeter that, defines an approximate “D” shape in cross section. The baffle 210 is attached to the interior of the hollow body 202 near the top of the hollow body 202. Consequently, the baffle 210 is attached to the hollow body 202 proximate the aperture exhaust 221.

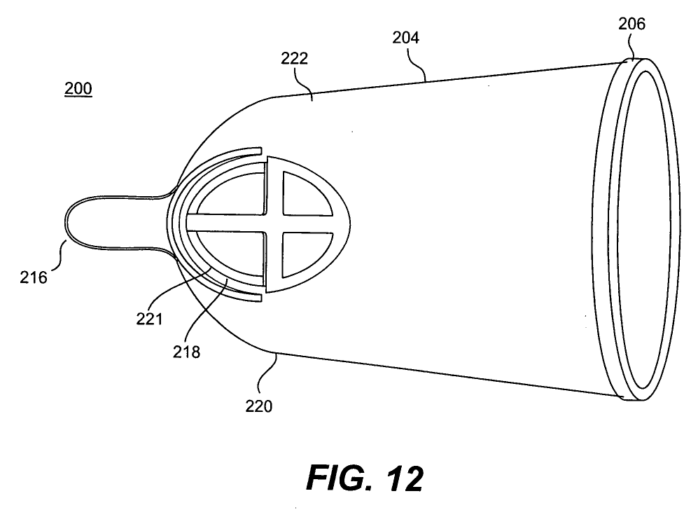

Referring to FIG. 12, a top view of the breath enhanced mouthpiece 200 is shown. Visible on the top of the breath enhanced mouthpiece 200 is the exhaust aperture 221 and an intake aperture 220. The exhaust aperture 221 is covered by a diaphragm 218 and communicates between the exterior of the breath enhanced mouthpiece 200 and the interior of the hollow body 202. The intake aperture 220 is disposed in the breath enhanced mouthpiece 200 proximate to where the baffle 210 attaches to the hollow body 202. Accordingly, the intake aperture 220 allows communication between the exterior of the breath enhanced mouthpiece 200 and the interior of the baffle 210.

The diaphragm 218 is fixed over the exhaust aperture 221 and the intake aperture 220. It should be noted that in this particular embodiment, the intake aperture 220 is configured so that gas carrier may flow from outside the breath enhanced mouthpiece 200 and through the intake aperture 220 into the interior of the hollow body 202, and in particular, into the interior of the baffle 210. The exhaust aperture 221 is also configured so that gas carrier may flow from inside the hollow body 202 and through the exhaust aperture 221 to the exterior of the breath enhanced mouthpiece 200.

The breath enhanced mouthpiece 200 also includes a drug delivery conduit 204 with a user interface 206. A protrusion 216 for storage or to facilitate mouthpiece use is attached to the hollow body 202 near the exhaust aperture 221.

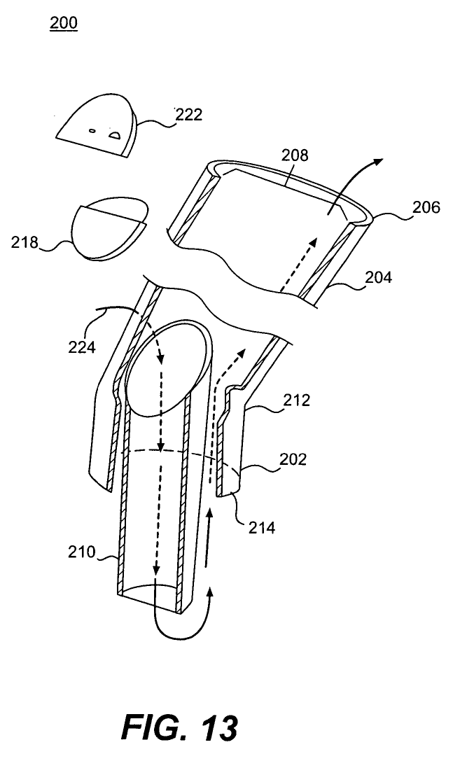

Referring to FIG. 13, the breath enhanced mouthpiece 200 is shown in ghost view. A delivery flow path 224 enters the breath enhanced mouthpiece 200 through the intake aperture 220. From the intake aperture 220, the delivery flow path 224 proceeds along the interior of the baffle 210. The delivery flow path 224 exits the baffle 210 near the bottom of the baffle 210 and proceeds along the exterior of the baffle 210 towards the union 212. At the union 212, the delivery flow path 224 enters the drug delivery conduit 204. The delivery flow path 224 proceeds along the interior of the drug delivery conduit 204 and exits the breath enhanced mouthpiece 200 at the drug delivery port 208.

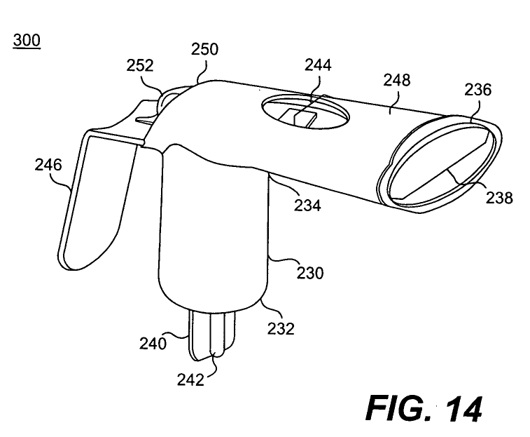

Referring to FIG. 14, another embodiment of the breath enhanced mouthpiece 300 is shown. The breath enhanced mouthpiece 300 includes a hollow body 230 attached to a drug delivery conduit 248 at a union 234. The hollow body 230 has a connection port 232 at its lower end and contains a baffle 240 running along a length of the interior of the hollow body 230. In this example, the baffle 240 is approximately planar with a ridge-like structure 242 running along the baffle's length.

The drug delivery conduit 248 has a mouthpiece 236 at one end and an inflow vent 250 at the opposite end. The mouthpiece 236 may also be referred to as a user interface. At the mouthpiece 236 is a drug delivery port 238 which is an opening which leads into the interior of the drug delivery conduit 248. On the top of the drug delivery conduit 248 is an exhaust vent structure 244. The inflow vent 250 includes a guard 252 configured to prevent objects such as, for example, a user's fingers from obstructing the inflow vent 250 during use. Proximate the guard 252 is attached a handle 246.

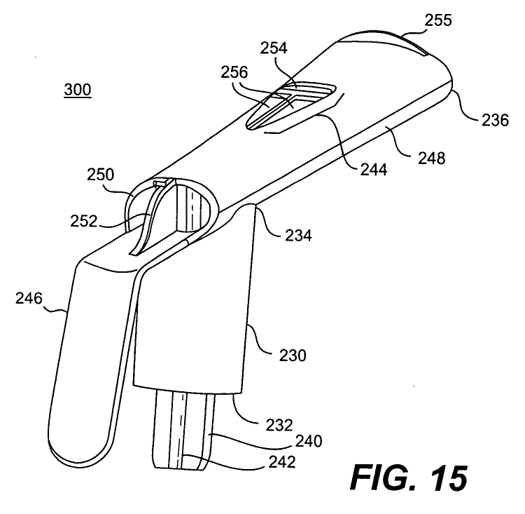

Referring to FIG. 15, the exhaust vent structure 244 includes two outlets 256, and a recess or aperture 254. The recess 254 is configured to attach a diaphragm to the top of the drug delivery conduit 248 which will selectively seal the two outlets 256. The recess 254 may also be an aperture leading into the interior of the drug delivery conduit 248. The breath enhanced mouthpiece 300 also includes ridges 255 on the drug delivery conduit 248 proximate the drug delivery port 236. The ridges 255 are configured to facilitate a user holding the mouthpiece 300 in his or her mouth.

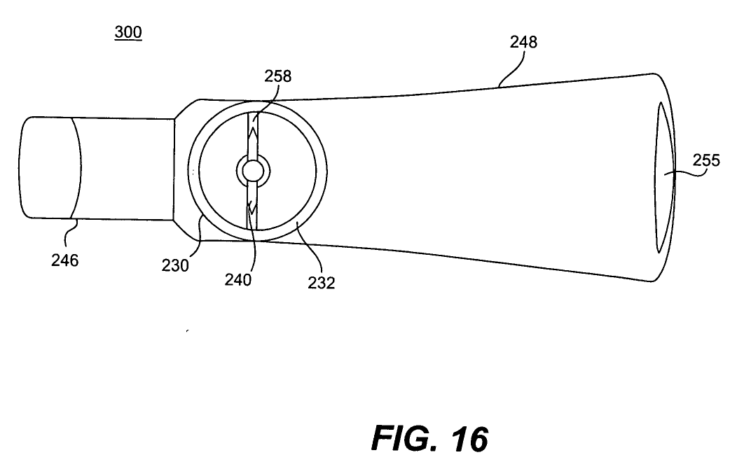

Referring to FIG. 16, a bottom view of the breath enhanced mouthpiece 300 is shown. Within the hollow body 232 is the baffle 240. The baffle 240 is attached to the interior of the mouthpiece 300 at a stop 258. The stop 258 is configured to control the degree of insertion of any fittings, including a nebulizer or aerosolization chamber, which may be attached to the mouthpiece 300 by sliding through the connection port 232 into the hollow body 230.

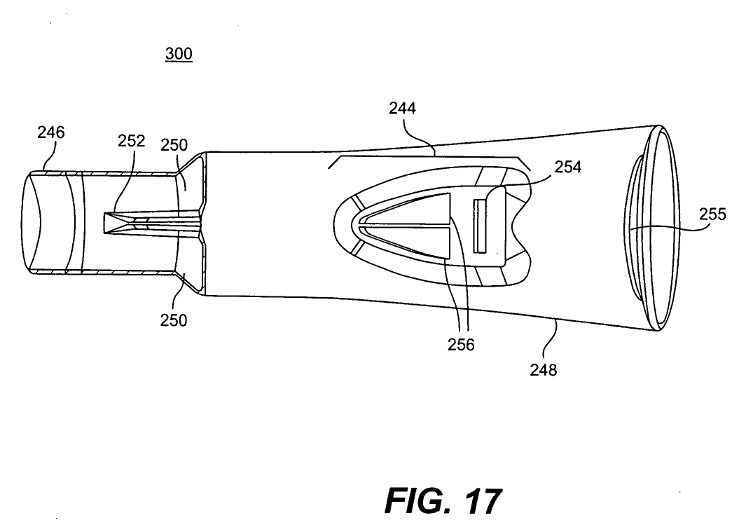

Referring to FIG. 17, a top view of the breath enhanced mouthpiece 300 is shown. The top of the breath enhanced mouthpiece 300 includes the exhaust vent structure 244. As shown, the recess 254 is a slot which is perpendicular to the long axis of the breath enhanced mouthpiece 300. Next to the recess 254 are the two outlets 256. It should be noted that the exhaust vent structure 244 may also be configured to receive fastening means for fastening a diaphragm thereto, such as a clip, pin, screw or adhesive.

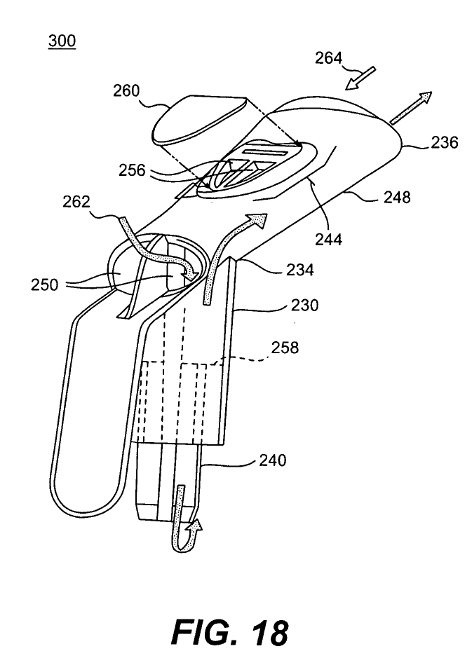

Referring to FIG. 18, a rear view of the breath enhanced mouthpiece 300 is shown. A diaphragm 260 may be attached to the exhaust vent structure 244. The diaphragm 260 is configured to seal against the exhaust vent structure 244 in such a manner as to form a one-way valve. The diaphragm 260 and the exhaust structure 244 are configured to cooperate with one another to allow gas carrier to exit out the top of the breath enhanced mouthpiece 300 while preventing gas carrier from entering therethrough. Additionally, the exhaust vent structure 244 may be configured to be manually opened and closed by a user during operation.

Still referring to FIG. 18, an intake flow path 262 is represented by the solid arrows, and an exhaust flow path 264 is represented by the hollow arrows. The intake flow path 262 enters the breath enhanced mouthpiece 300 at inflow vent 250. After entering the inflow vent 250, the intake flow path 262 travels down the hollow body 230 towards the bottom of the baffle 240. At the bottom of the baffle 240 the intake flow path 262 makes an approximately 180 degree turn and proceeds up the hollow body 230 on the opposite side of the baffle 240 towards the union 234. At the union 234, the intake flow path 262 enters the drug delivery conduit 248 and proceeds towards the drug delivery port 236 to exit out of the breath enhanced mouthpiece 300 at the drug delivery port 236.

The exhaust flow path 264 enters the breath enhanced mouthpiece 300 at the drug delivery port 236. The exhaust flow path 264 travels along a portion of the drug delivery conduit 248 to exit out the top of the breath enhanced mouthpiece 300 through the outlets 256 of the exhaust structure 244.

The breath enhanced mouthpiece 10 is used to illustrate operation of the various embodiments of the invention. Referring back to FIG. 1, the breath enhanced mouthpiece 10 is attached to a nebulizer at the connection port 40. The nebulizer contains a supply of a drug or other substance which will be mixed or entrained in the gas carrier which passes through the aerosolization chamber area.

When a user puts the user interface or mouthpiece 18 into his mouth and inhales, gas carrier is drawn in through the intake 34 at the top of the hollow bodied 12 and travels down the baffle 42 shaped like a conduit and out its lower end. After exiting the baffle 42, the gas carrier flow enters the aerosolization chamber or nebulizer area where it is mixed with the drug or material to be delivered. The gas carrier now entrained with the drug or material to be delivered, passes out of the aerosolization area then travels back up the hollow body 12 along the exterior of the baffle 42. The gas carrier and drug or material combination then passes through the union inlet 46 and enters the drug delivery conduit 14. The gas carrier and drug or material then travel towards the drug delivery port 16. The gas carrier and drug or material then exit the breath enhanced mouthpiece 10 at the drug delivery port 16 and enters the user's mouth to find its way into the user's respiratory system.

The other embodiments work in a similar manner, with some of the embodiments relying on a diaphragm in combination with a vent or vents to direct gas carrier along certain flow paths. For example, the inlet vent 112 shown in FIG. 8 is configured so that gas carrier pressure caused by inhalation opens the inlet vent 112 and closes outlet vents 120. Thus inhalation causes the inlet vent 112 to allow gas carrier to enter the delivery flow path 130 and proceed towards the nebulization area and into the patient's mouth. During exhalation, gas carrier pressure causes the inlet vent 112 to close, and outlet vents 120 to open and allow gas carrier in the exhaust flow path 131 to exit the mouthpiece 100.

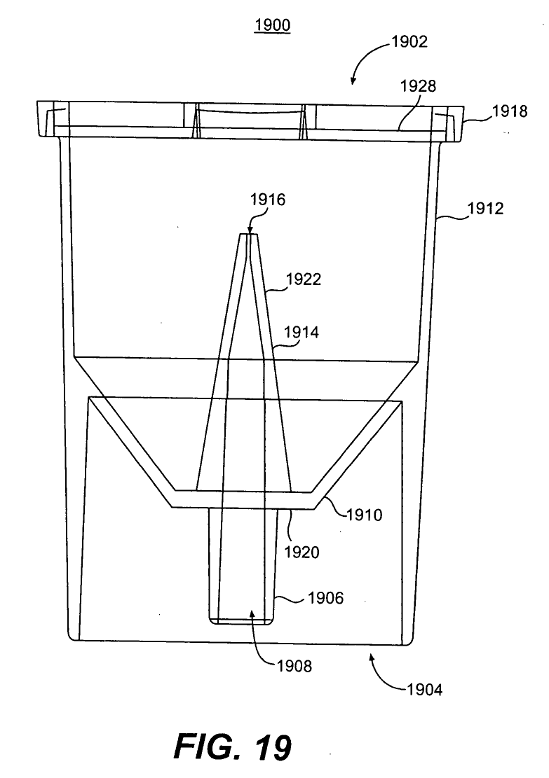

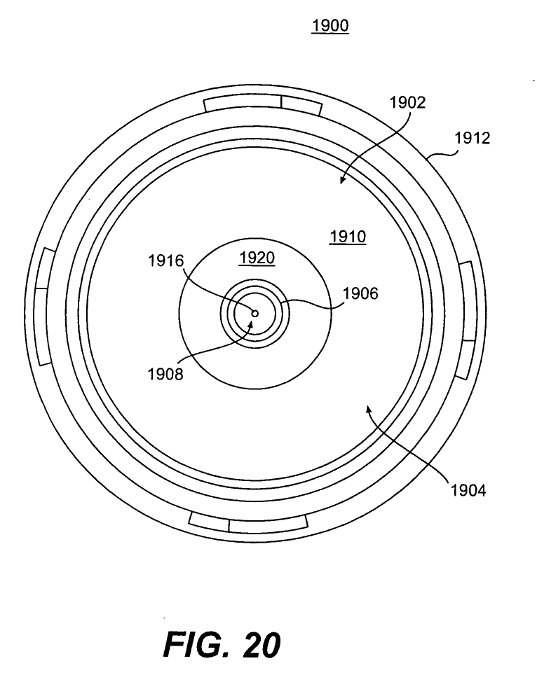

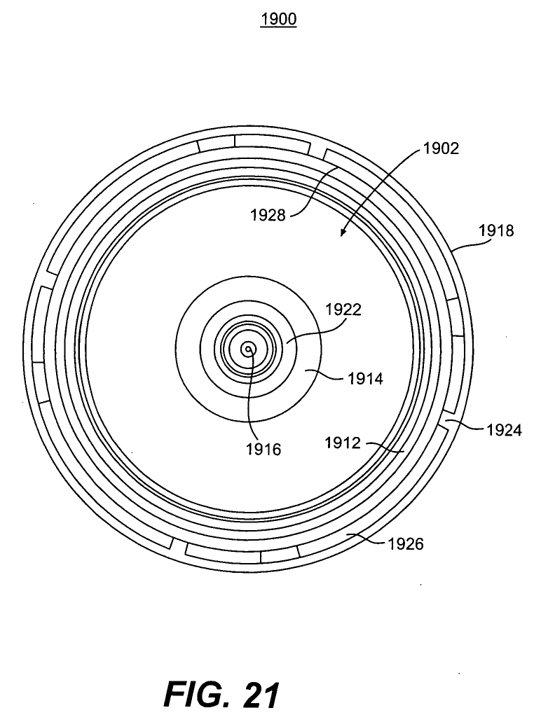

FIGS. 19-21 illustrate a hollow body according to principles of the invention. More particularly, FIG. 19 illustrates a side view of a hollow body according to principles of the invention. FIG. 20 illustrates a bottom view of a hollow body according to principles of the invention. FIG. 21 illustrates a top view of a hollow body according to principles of the invention. The hollow body 1912 will now be described in greater detail.

As illustrated in hollow body 1912 includes a cap opening 1902 and a connection opening 1904. Hollow body 1912 has located within it an gas carrier port 1906 that defines an gas carrier intake 1908. An angled cone support 1910 and flat cone support 1920 substantially form a barrier between the cap opening 1902 and the connection opening 1904. Angled cone support 1910 connects to the hollow body 1912. Gas carrier port 1906 is connected to flat cone support 1920 to provide a passage through the flat cone support.

The gas carrier intake 1908 continues through a body cone 1914 which is connected to the flat cone support 1920. The body cone 1914 includes a cone gas carrier port 1916 defined by a cone nozzle 1922 that connects the gas carrier intake to the cap opening 1902. According to an embodiment of the invention, a source of gas carrier, such as a hose connected to an gas carrier compressor, is attached to gas carrier port 1906. Gas carrier passes through the gas carrier intake 1908 and out the body cone 1914 through the cone gas carrier port 1916.

Referring now to FIGS. 20 and 21, cap opening 1902 is defined by body rim 1918. Body rim 1918 includes one or more connection locks 1924 which may interact with a cap. According to an embodiment of the invention, a rim notch 1926 is located between each of the connection locks 1924. The cap may be placed on the rim 1918 of the hollow body 1912 so that a connection portion of the cap may be inserted into a rim notch 1926 and rest on a rim ledge 1928. The cap may then be rotated so that the connection portion is rotated to engage the connection lock 1924. As shown, the connection portion may be located under the connection lock 1924 to secure the cap to the hollow body 1912. Other connection mechanisms may also be used.

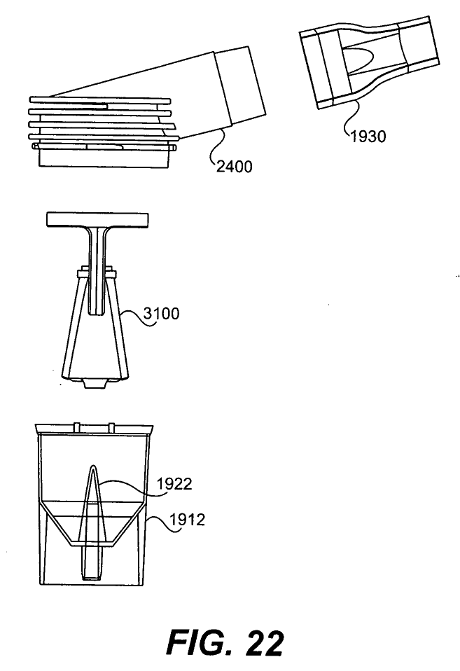

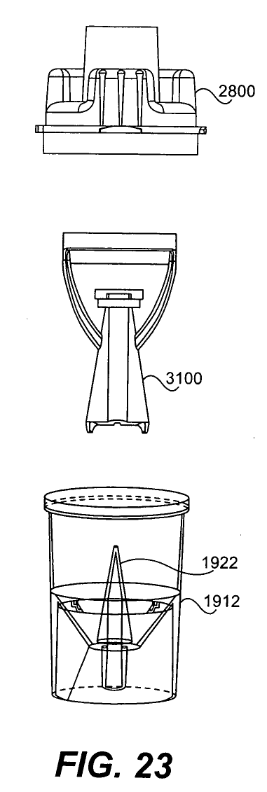

FIGS. 22 and 23 illustrate a hollow body in use according to principles of the invention. FIG. 22 illustrates an exploded view of the hollow body 1912, an insert 3100, a breath cap 2400 and a mouthpiece 1930. FIG. 23 illustrates an exploded view of the hollow body 1912, the insert 3100, and a ventilator cap 2800. Insert 3100, breath cap 2400 and ventilator cap 2800 will now be described in greater detail.

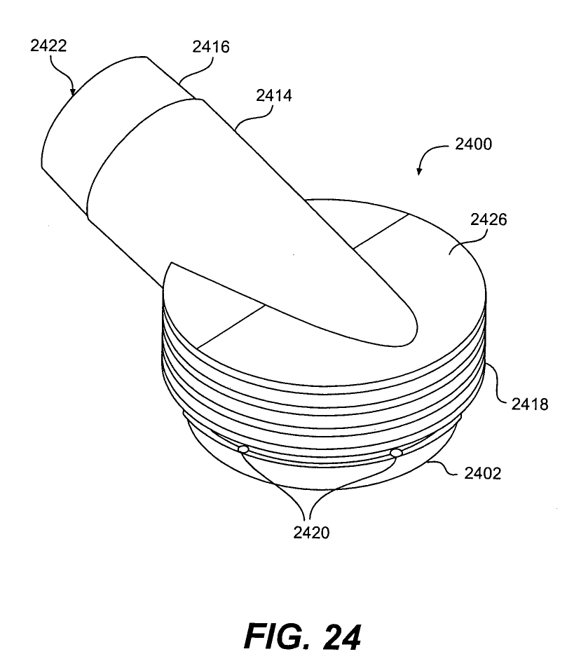

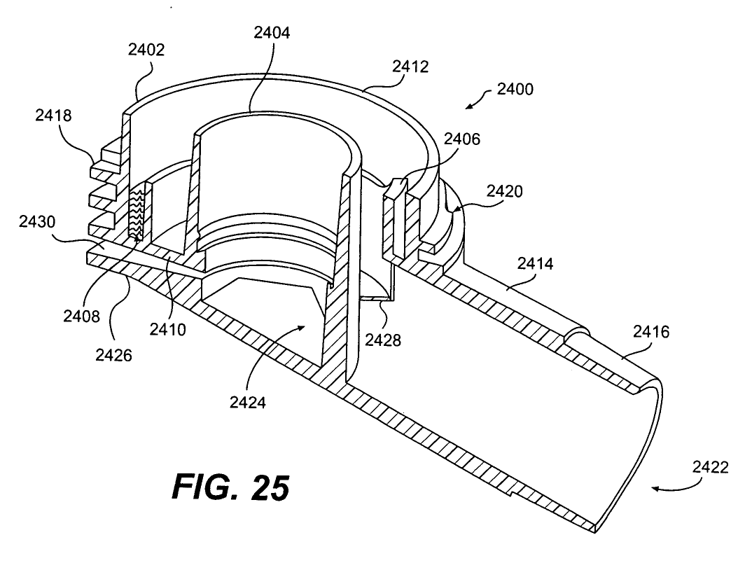

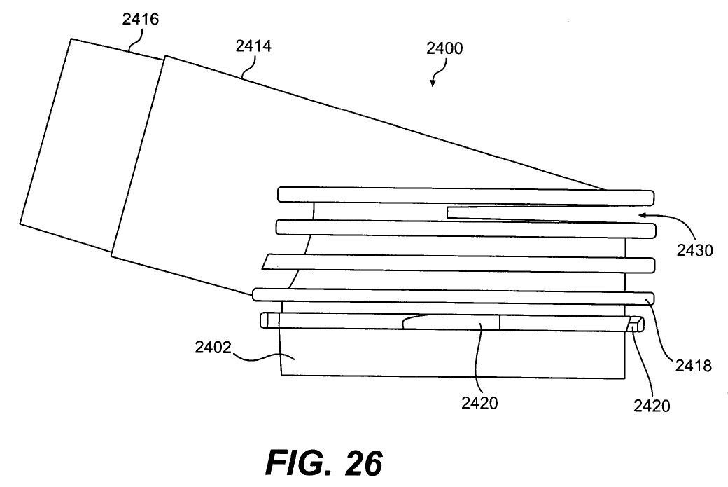

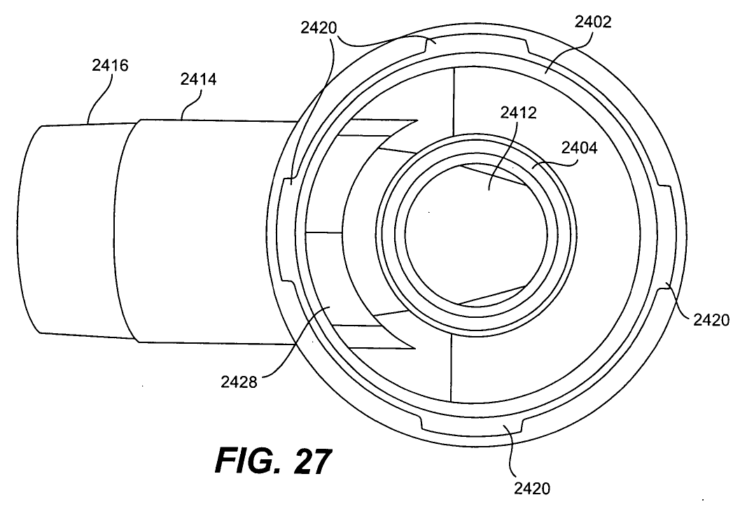

FIGS. 24-27 illustrate a breath cap according to principles of the invention. More specifically, FIG. 24 illustrates a perspective view of the breath cap. FIG. 25 illustrates a cut-away view of the breath cap from the bottom. FIG. 26 illustrates a side view of the breath cap. FIG. 27 illustrates a bottom view of the breath cap. The breath cap will now be described in greater detail.

Referring to FIG. 24, which is a top view of a breath cap, generally depicted as reference numeral 2400, is provided according to principles of the invention. Cap 2400 has a delivery conduit 2414 protruding from one side. As illustrated, delivery conduit 2414 may be at an angle relative to the plane of the top 2426 of the cap 2400. Cap 2400 also includes outer wall 2402 with outer fins 2418. A connection piece 2420 connects the cap 2400 to a hollow body, such as the hollow body 1912 of FIG. 19. While connection piece 2420 is a protrusion that may be inserted into a cavity in the hollow body 1912, it is understood that other types of connections may also be used.

The delivery conduit 2414 and mouthpiece junction 2416 of cap 2400 include a hollow delivery opening 2422. The mouthpiece junction 2416 provides a junction for joining a mouthpiece (not shown) to delivery conduit 2414. The junction 2416 may provide the ability to permit various types of mouthpieces to be used for any given cap 2400.

Now referring to FIG. 25, a cut away view of the breath enhanced cap 2400 according to principles of the invention is provided. Cap 2400 includes an outer wall 2402, an inner wall 2404 and an intermediate wall 2406. According to an embodiment of the invention, outer wall 2402, inner wall 2404 and intermediate wall 2406 may be substantially cylindrical. Outer wall 2402 and intermediate wall 2406 are arranged with cap 2400 to form an outer trough 2408. Intermediate wall 2406 and inner wall 2404 are arranged with cap 2400 to form an inner trough 2410. FIG. 25 illustrates outer wall 2402 and inner wall 2404 as substantially the same height relative to the cap top 2426, while intermediate wall 2406 is a shorter height in at least some portion. However, it is understood that different wall heights can also be used.

According to an embodiment of the invention, inner trough 2410 may have a trough opening 2428 connecting inner trough 2410 to the delivery opening 2422 of delivery conduit 2414. Further, gas carrier flow passage 2412 connects via a gas carrier flow opening 2424 to delivery opening 2422. Outer trough 2408 may be substantially uniform, with no openings in outer wall 2402 or intermediate wall 2406 and therefore no connection with the delivery opening 2422 of delivery conduit 2414.

Referring to FIGS. 25 and 26, a cap vent 2430 is located in outer wall 2402. As illustrated in FIG. 26, the cap vent 2430 is located between two outer wall fins 2418. Cap vent 2430 is an opening that connects gas carrier flow passage 2412 to the outside. Cap vent may be used to vent gas carrier from gas carrier flow passage 2412 to reduce the pressure of gas carrier received by a user through delivery opening 2422. Further, when a user exhales into the mouthpiece through delivery opening 2422, gas carrier may flow through cap vent 2430 to facilitate exhalation by the user.

With reference to FIG. 19, gas carrier passes through the hollow body 1912 and a liquid solution or suspension which may contain an active ingredient, such a drug, is aerosolized into the gas carrier flow (as described previously, the aerosolized solution/suspension is further referred to as gas carrier). The gas carrier travels through the hollow body 1912 into gas carrier flow passage 2412, inner trough 2410 and outer trough 2408. As there are no outlets in outer trough 2408, the gas carrier is halted at outer trough 2408. However, the gas carrier moves through gas carrier flow passage 2412 and into delivery opening 2422 via the gas carrier flow opening 2424. The gas carrier also moves through inner trough 2410 into delivery opening 2422 via the trough opening 2428. The gas carrier is then delivered via delivery opening 2422 and a mouthpiece to the user.

Intermediate wall 2406 may prevent the liquid from entering the mouthpiece if the nebulizer is shifted or moved. According to an embodiment of the invention, cap 2400 may be arranged relative to a hollow body 1912 such that the liquid is substantially located at one end of the hollow body 1912 and the cap 2400 is located at the other end of the hollow body 1912. The top 2426 is located above the liquid, leaving inner trough 2410 and outer trough 2408 open to the liquid. In this arrangement, the user draws gas carrier through the mouthpiece to inhale the gas carrier. When the hollow body 1912 is tilted and inverted, the liquid may move toward the cap 2400. Some or all of the liquid may be retained in outer trough 2408. This may prevent the liquid from entering the delivery opening 2422 of the delivery conduit 2414.

According to principles of this aspect of the invention, an outer trough 2408 formed by intermediate wall 2406 and an outer wall 2402 may contain the liquid in the event that the hollow body is tilted or inverted, thereby precluding the user from inadvertently receiving the liquid directly through the mouthpiece. In addition, the liquid may be prevented from entering the gas carrier flow opening 2424 and thus being expelled from the nebulizer. These advantages may be particularly desirable when the liquid is concentrated or expensive. Specifically, for example, the nebulizer may be used for delivery of a drug, which may be placed in the nebulizer, such as the hollow body 1912, in a concentrated form, to a user via the gas carrier flow. However, it may not be desirable to administer such a concentrated dose of the drug directly to the user. Therefore, if the drug is contained in the outer trough 2408, it may preclude the user from being inadvertently exposed to the concentrated drug through the mouthpiece. Alternatively, the drug may be expensive, and by containing the drug in the outer trough 2408, loss of the drug may be reduced or prevented if the hollow body 1912 is dropped or inverted, thereby reducing the expense of supplying an additional dose of the drug.

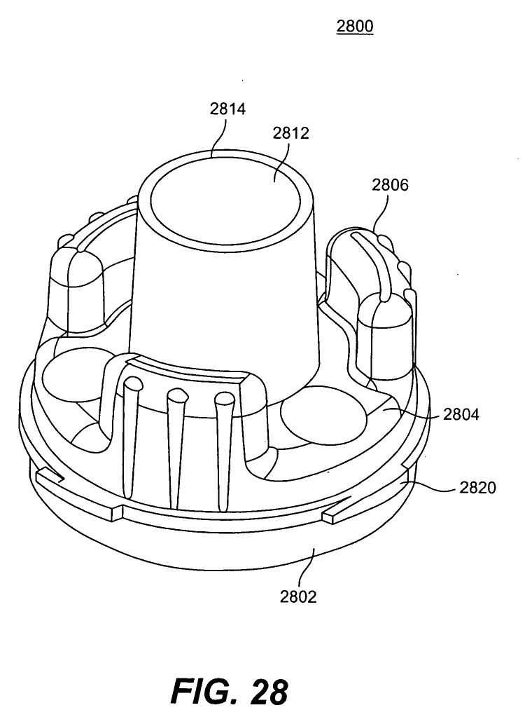

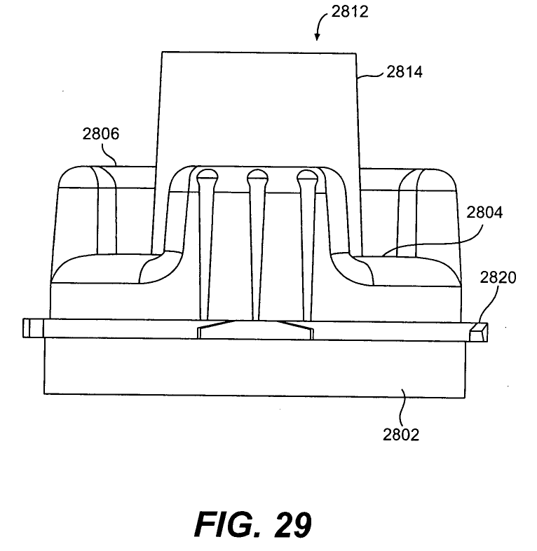

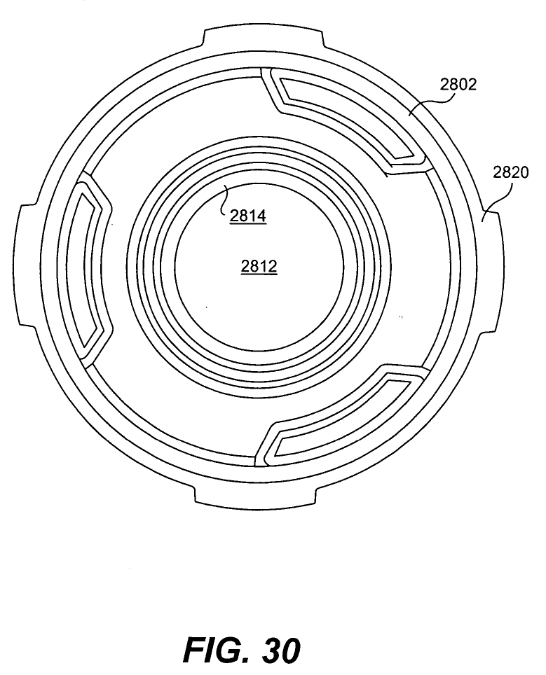

FIGS. 28, 29 and 30 illustrate a ventilator cap according to principles of the invention. More specifically, FIG. 28 is a perspective view of ventilator cap 2800, FIG. 29 is a side view of ventilator cap 2800, and FIG. 30 is a bottom view of ventilator cap 2800. As illustrated in FIG. 23, the ventilator cap 2800 may connect with a hollow body 1912 to form a portion of a nebulizer system. The ventilator cap 2800 includes an outer wall 2802 and a cap top 2804. With reference to the hollow body 1912 of FIG. 19, outer wall 2802 may fit within the hollow body 1912. The connection pieces 2820 of the ventilator cap 2800 may rest on the rim ledge 1928. The ventilator cap 2800 may be rotated so that the connection pieces of the ventilator cap 2800 engage the connection lock 1924 at the body rim 1918. Other connection mechanisms may also be used.

Ventilator cap 2800 includes a delivery conduit 2814 that defines an gas carrier flow passage 2812. Gas carrier passing through the hollow body 1912 passes through ventilator cap 2800 and through gas carrier flow passage 2812 into a ventilator system (not shown). Liquid contained within the hollow body 1912 enters the gas carrier stream and is delivered through the gas carrier flow passage of the ventilator cap 2800. The aerosolized liquid is thereby introduced into the gas carrier stream of the ventilator and administered to the user. Ventilator cap 2800 allows the hollow body 1912 to be used to administer a liquid solution or suspension containing an active ingredient, such as a drug or other chemical compound, to a user on a ventilator using inhalation techniques. This may allow patients to receive appropriate treatment regimen even if the user is otherwise unable to participate in the treatment regimen

Ventilator cap 2800 includes protrusions 2806 that protrude from the cap top 2804. While FIGS. 28 and 29 illustrate three protrusions 2806, it is understood that more or fewer protrusions 2806 may be used. According to an embodiment of the invention, protrusions 2806 may cooperate with delivery conduit 2814 to secure any ventilator attachments to the ventilator cap 2800. Thus, an attachment (not shown) to the ventilator may be inserted around the delivery conduit 2814. The delivery conduit 2814 may engage the inside of the attachment while the protrusions 2806 may engage the outside of the attachment, thereby securing the attachment to ventilator cap 2800. Other securing mechanisms may also be used.

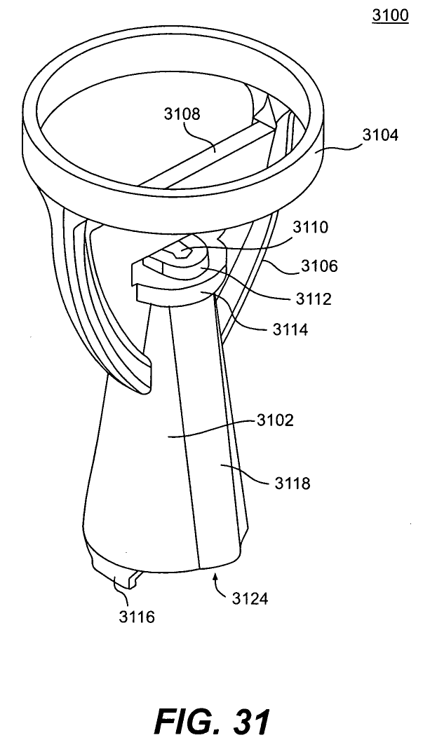

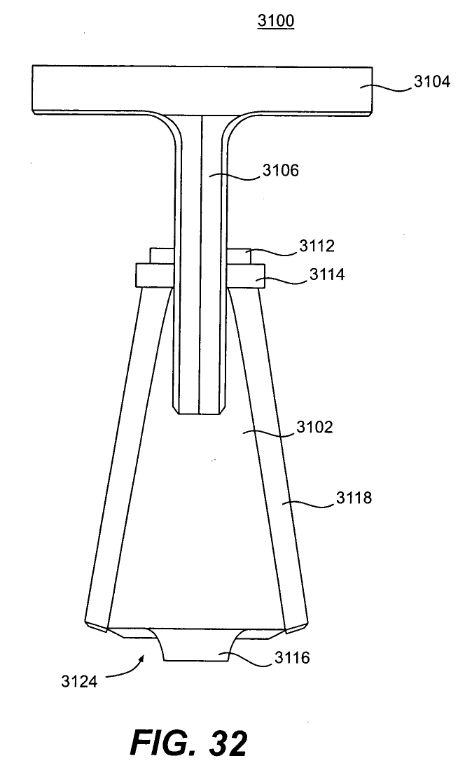

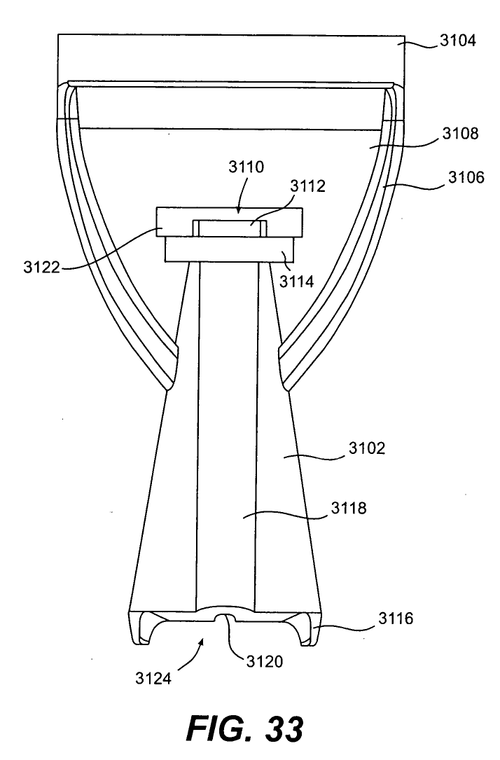

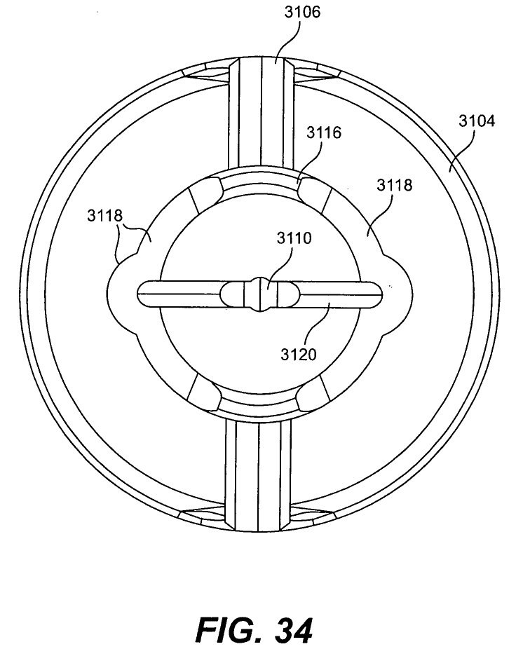

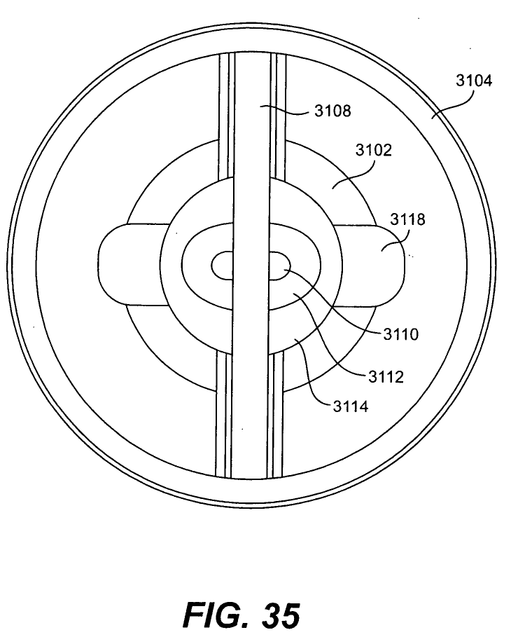

FIGS. 31-35 illustrate an aerosol insert for the hollow body according to principles of the invention. More specifically, FIG. 31 illustrates a perspective view of aerosol insert 3100. FIG. 32 illustrates a side view of aerosol insert 3100. FIG. 33 illustrates a front view of aerosol insert 3100. FIG. 34 illustrates a bottom view of aerosol insert 3100. FIG. 35 illustrates a top view of aerosol insert 3100. Aerosol insert 3100 will now be described in greater detail.

With reference to FIGS. 31-35, aerosol insert 3100 includes an aerosol cone 3102 with a wick protrusion 3108. A cone support 3116 is located at one end of the aerosol cone 3102. According to an embodiment of the invention, two wick protrusions 3108 are located on opposite sides of the aerosol cone 3102, while two cone supports 3116 are located on opposite sides of the aerosol cone 3102, so that a cone support 3116 is located between the wick protrusions 3108.

Aerosol cone 3102 is hollow with a cone gas carrier passage 3124 located therein. Within the cone gas carrier passage 3124, wicks 3120 are located opposite of the wick protrusions 3118. Wicks 3120, which are illustrated in FIG. 34 as indentations on the inside of aerosol cone 3102, are located along the entire length of the aerosol cone 3102. The wicks 3120 lead from the bottom of the aerosol cone 3102 to the gas carrier passage 3110.

As illustrated in FIGS. 22 and 23, aerosol insert 3100 is placed in hollow body 1912 so that aerosol cone 3102 is placed over body cone 1922. The aerosol cone 3102 is seated so that the cone gas carrier passage 3124 is substantially seated on body cone 1922. Cone supports 3116 may support the aerosol insert 3100 by contacting the flat cone support 1920. In this configuration, any liquid retained in by the flat cone support 1920 and the angled cone support 1910 may be directed via the wicks 3120, such as by a capillary action in connection with the wicks 3120 and the body cone 1914 up toward the gas carrier passage 3110. Gas carrier passage 3110 is aligned with cone gas carrier port 1906 of the body cone 1914. As gas carrier passes through cone gas carrier port 1906 of the body cone 1914, the liquid is aerosolized into the gas carrier stream. This gas carrier is then directed to the user.

Aerosol insert 3100 also includes a support ring 3104 located above the aerosol cone 3102 and supported by arms 3106. An arm support 3108 is located between arms 3106. According to an embodiment of the invention, arm support 3108 is a substantially solid piece that connects arms 3106 to each. Further, arm support 3108 connects to the arms 3106 from the point where the arms 3106 connect to the aerosol cone 3102 to a point somewhere below where the arms 3106 connect to the support ring 3104. Support ring 3104 may allow a user to handle the aerosol insert 3100 and place it or remove it from a hollow body 1912. Other types of mechanisms for handling the aerosol insert 3100 may also be used.

A cone top 3114 is located at the top of aerosol cone 3102, and is connected to arm support 3108. An arm support opening 3122 is located in the arm support 3108. As illustrated in FIG. 33, the arm support opening 3122 is located above the cone top 3114 so that the bottom edge of arm support opening 3122 is even with the top of cone top 3114. An gas carrier outlet 3112 defining gas carrier passage 3110 is located on top of cone top 3114. According to an embodiment of the invention, the height of gas carrier outlet 3112 is less than the height of arm support opening 3122. Gas carrier flows through gas carrier passage 3110 and contacts arm support 3108. The gas carrier is redirected by the arm support 3108, thereby reducing the force of gas carrier flow through the gas carrier passage 3110. The gas carrier is redirected to the hollow body 1912 and then through the opening within the cap (such as a breath cap 2400 or ventilator cap 2800).

Suitable active ingredients of the invention may possess one or more of the following activities which may be used in any combination, for example, analgesics, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immuriological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators and xanthines. The active ingredients are commercially available and/or may be prepared by techniques known in the art.

According to an embodiment of the invention, the active ingredient may be formulated as a liquid solution, suspension, aerosol propellant or dry powder loaded into a suitable dispenser for administration, such as the nebulizer of the invention. Furthermore, the active ingredient of the invention may be used in combination with at least one pharmaceutically acceptable carrier or excipient. Acceptable carriers or excipients are non-toxic, aid administration and do no adversely affect the therapeutic benefit of the compound. Specifically, for example, the excipient may be a gaseous excipient that is generally available to one of skill in the art.

Note that although the above example illustrates vent operation by gas carrier pressure created during inhalation and exhalation, the vents may also be actuated directly by the user, such as through opening and closing the vents with his fingers. Additionally, the vents may be operated by any vent operation mechanism well know in the art, including, for example, electronic actuation, spring actuation, etc.

It should be noted that embodiments of the invention are of dimensions scaled to facilitate use by a variety of patients of different sizes and physical conditions. Thus, embodiments are typically sized to be easily grasped, easily inserted into, and easily held in a patient's mouth.

What is claimed is:

1. A breath enhanced device comprising:
a delivery conduit;
a cap; and
a plurality of cylinders, wherein said plurality of cylinders are attached to said cap such that when said cap is attached to a container, a liquid within the container is prevented from flowing through said drug delivery conduit when said cap is in a first position.

2. The device of claim 1, wherein said plurality of cylinders comprise:
an inner cylinder;
an outer cylinder; and
an intermediate cylinder located between said outer cylinder and said inner cylinder.

3. The device of claim 2, where in said inner cylinder and said outer cylinder have substantially the same height in relation to said cap, and said intermediate cylinder has a different height in relation to said cap.

4. The device of claim 1, further comprising a hollow chamber having two openings, wherein at least one of said plurality of cylinders attaches said cap to said hollow container at said at least one opening wherein the liquid is contained in said hollow chamber.

5. The device of claim 4, wherein said second opening is attached to a gas carrier flow such that at least a portion of the liquid is aerosolized into the gas carrier flow and wherein said aerosolized gas carrier flow is delivered to a subject through said delivery conduit.

6. The device of claim 1, wherein the liquid is a drug.

7. The device of claim 1, wherein the liquid is a solution.

8. The device of claim 7, wherein said solution includes an active ingredient.

9. The device of claim 8, wherein the active ingredient is at least one selected from the group consisting of an anti-inflammatory and an anti-infective.

10. A device for delivery of a compound comprising:
a hollow body having an airflow inlet for receiving a gas carrier flow and an opening, said hollow body containing a liquid;
an aerosol insert interacting with said hollow body to aerosolize the liquid into the gas carrier flow, wherein said aerosol insert is separate and removable from said hollow body; and
a cap including a delivery conduit for directing the aerosolized liquid and the gas carrier, wherein said cap is separate and removable from said hollow body and said aerosol insert.

11. The device of claim 10, wherein said cap directs the aerosolized liquid and the gas carrier to a mouthpiece.

12. The device of claim 10, wherein said cap directs the aerosolized liquid and the gas carrier into a ventilator gas carrier flow.

13. The device of claim 10, wherein said cap directs the aerosolized liquid and the gas carrier flow into a subject's lungs.

14. The device of claim 10, wherein the liquid is a drug.

15. The device of claim 10, wherein the liquid is a solution.

16. The device of claim 15, wherein said solution includes an active ingredient.

17. The device of claim 16, wherein the active ingredient is at least one selected from the group consisting of an anti-inflammatory and an anti-infective.

18. A device for delivery of a compound comprising:
a container holding the compound, said container including an insert and receiving a gas carrier and wherein the gas carrier interacts with said insert to form a mixture of the gas carrier and the compound;
a cap having a first cap opening and a second cap opening, said first cap opening engaging said container holding the compound; and
a connector having:
a first container opening connected to said second cap opening; and
a second container opening cooperating to direct the mixture.

19. The device of claim 18, wherein said connector directs the aerosolized liquid and the gas carrier to a mouthpiece.

20. The device of claim 18, wherein the gas carrier is received from a ventilator.

21. The device of claim 18, wherein said connector directs the mixture into a subject's lungs.

22. The device of claim 18, wherein the liquid is a drug.

23. The device of claim 18, wherein the liquid is a solution.

24. The device of claim 23, wherein said solution includes an active ingredient.

25. The device of claim 24, wherein the active ingredient is at least one selected from the group consisting of an anti-inflammatory and an anti-infective.
